(19) United States
(12) Patent Application Publication
(10) Pub. No.: US 2011/0178607 A1
(43) Pub. Date: Jul. 21, 2011

(54) Orthopedic Prosthesis

(75) Inventor: Christiaan Rudolf Oosthuizen, Johannesburg (ZA)

(73) Assignee: Christiaan Rudolf Oosthuizen, Johannesburg (ZA)

(21) Appl. No.: 13/055,412

(22) PCT Filed: Jul. 24, 2009
(86) PCT No.: PCT/IB09/53224

§ 371 Date: Apr. 5, 2011

(30) Foreign Application Priority Data
Jul. 24, 2008 (ZA) 2008/05596

(51) Int. Cl. A61F 2/38

(52) U.S. Cl. 623/20.35

(57) ABSTRACT

According to one aspect of the present invention, there is provided an orthopedic prosthesis (10) suitable for resurfacing chondral deficient surface areas in knee joints, including a tibial component (30) defining a tibial articular bearing surface (50), a femoral component (20) defining a femoral articular bearing surface (130) and a spacer bearing (40) disposed therebetween, the femoral component further including an elliptical body having an anterior member (110) and a posterior member (120), and an internal femoral attachment surface (140), the femoral articular bearing surface and the internal femoral attachment surface having a substantially uniform cross-sectional curvature at any point along the length of the anterior member, thereby providing an increased radius of the internal femoral attachment surface and an increased cross sectional line of fixation to bony tissue.

### FIELD OF THE INVENTION

This invention relates to an orthopedic prosthesis. More particularly, this invention relates to an improved resurfacing prosthesis of the knee, and linked thereto an improved surgical approach to knee replacements.

### BACKGROUND OF THE INVENTION

Various types of knee prostheses/prosthetic joints are known in the field of orthopedic surgery, and these are widely used by orthopedic surgeons. In particular, knee prostheses have been developed since the pioneering efforts of Sir John Charnely, and in particular his revolutionary hip replacement techniques introduced during the 1960's.

In order to understand the background to the present invention and the rationale behind knee replacements or revisions, it is necessary to briefly describe the basic anatomy of a knee. A knee joint is formed by the articulation of a femur with a tibia and fibula. The femur has at its substantially cuboid lower extremity two oblong eminences, a lateral and medial condyle. These condyles are separated from each other by a smooth articular depression called a patellar surface. Behind the patellar surface is a deep notch known as an intercondylar fossa.

The medial condyle is longer and, when the femur is held perpendicular to the normal, extends to a lower level as opposed to the lateral condyle. The condyles have a layer of articular cartilage that extends substantially across the anterior, inferior and posterior surfaces of the condyles. The lower and posterior surfaces of these condyles constitute the tibial surfaces for articulation with the corresponding plateaus of the tibia and menisci.

The menisci are two c-shaped fibrocartilage discs disposed between the femur and the tibia. The menisci comprise a lateral meniscus and a medial meniscus, and function to support the femur and tibia, to provide lubrication therebetween (with the assistance of synovial fluid), as well as to cushion for stresses.

Several ligaments function to hold the knee joint in place, and include medial collateral ligaments and lateral collateral ligaments. Also present are the anterior cruciate and the posterior cruciate ligaments.

Problems may arise with knee joints due to various factors, including injury, old age, and genetic predisposition to degenerative diseases. Osteoarthritis can occur after years of normal use of the knee, causing the menisci to crack and wear away. Alignment problems such as bow-legs and so-called “knock knees” may speed up this wear process. A further disease that may lead to problematic knees is rheumatoid arthritis. This is an inflammatory joint disease that destroys the menisci. In both conditions, once the menisci are destroyed or worn away, as the case may be, the femoral condyles will rub against the tibial plateaus, leading initially to pain and swelling and a limited range of movement, and ultimately to a fusing of the joint. Typically, the medial meniscus wears away before the lateral meniscus. This is known as medial compartment arthritis.

The purpose of a knee prosthesis is to provide pain relief, to increase the (presently) restricted range of movement, and to attempt to return a person suffering from a problematic knee to as normal a life as possible. Surgical insertion of the prosthesis is required and is resorted to where no other remedies are available or where such remedies have been exhausted.

Two techniques are available, the first involving an invasive total knee replacement whereby prosthetic devices are surgically attached to both the medial and lateral femoral condyles in aggravated presentations, and a second less-invasive technique known as a partial or unicompartmental knee replacement, whereby a prosthesis is surgically attached to the medial condyle only. In both instances, a corresponding tibial component is surgically attached to the tibial plateau, and a plastic spacer/bearing is inserted therebetween, in order to mimic the functioning of the menisci as these are removed.

A number of prior art prosthesis are known, the most notable of which is the so-called mobile bearing Oxford® Unicompartmental Knee (“the Oxford knee”). As described above, this prosthesis consists of a femoral component, a tibial component and a plastic spacer/bearing therebetween. The spacer acts as an articular bearing surface between the tibial and femoral components, respectively. The Oxford knee may have a disadvantage of a limited range of movement and a potential for bearing insert dislocation when the knee is flexed beyond approximately 130°. Furthermore, axial rotation of the knee while the knee is flexed to substantially 90°, or extended to 0°, may result in pronounced spacer/bearing insert overhang, and may further restrict the practical utility of the Oxford knee.

Furthermore, the size and thickness of the femoral component of prior art prostheses, including the Oxford knee, requires a large amount of bone to be removed from the femoral condyle in order to accommodate the prosthesis. This is hardly suitable and because of the limited lifespan of the prior art prostheses (10-15 years), a replacement of same may be indicated at that time. Where a substantial amount of bone had been previously removed, a more invasive replacement of the implant may be indicated and a total knee replacement may become necessary, or a revision type of bone augmented replacement.

The challenges in knee replacement prosthetic devices are in summary threefold: to limit the problems of wear, loosening of the prosthesis in use and a post-surgical loss of bone. These represent some of the additional disadvantages associated with the prior art prostheses.

At present, the less invasive uni-compartmental knee replacement is invariably the first step in treating the knee conditions described above. Such prostheses unfortunately usually last for only about 15 years on average, and thereafter a so-called full knee replacement is required. The latter prostheses also last for only about 15 years. Accordingly, one requirement in this field is for knee prostheses to last for longer periods. Another approach to this problem is that, with elderly people living increasingly longer lives, especially in first world countries, an improved technique in such knee procedures could assist such patients in ensuring that such knee prostheses last for an extended period covering the entire remaining lifetime of such patients (which is expected to increase in the future).

### OBJECTS OF THE INVENTION

It is an object of the present invention to provide a new and improved orthopedic prosthesis that overcomes, at least partially, the disadvantages associated with the prior art, typically in cases of anteromedial arthritis.

It is also an object of the present invention to provide an improved knee prosthesis that involves an inventive step relative to the prior art.

### SUMMARY OF THE INVENTION

According to one aspect of the present invention, there is provided an orthopedic prosthesis suitable for resurfacing chondral deficient surface areas in knee joints, including a tibial component defining a tibial articular bearing surface, a femoral component defining a femoral articular bearing surface and a spacer bearing disposed therebetween, the femoral component further including:
an elliptical body having an anterior member and a posterior member, and an internal femoral attachment surface;
the femoral articular bearing surface and the internal femoral attachment surface having a substantially uniform cross-sectional curvature at any point along the length of the anterior member, thereby providing an increased radius of the internal femoral attachment surface and an increased cross sectional line of fixation to bony tissue.

The elliptical body may be a substantially c-shaped body when viewed from the side. The elliptical body may further be a uniradial body and may provide a free range of movement of approximately 0° to 130° in situ.

The cross-sectional thickness of the anterior member may be less than 3 mm thick, and preferably 2 mm thick, at any point along the anterior member. This feature requires less invasive surgery that involves the removal of less bone from the femoral (medial or lateral) condyle.

Also, according to the invention, there may be provided that the posterior member may define a flattened interior surface, thereby defining a posterior fixation point.

The invention may further provide that the femoral internal surface further defines a line of attachment extending across the substantially outer circumferential edge of the femoral internal surface.

The femoral component may have a raised medial edge relative to its lateral edge for movably abutting a corresponding formation provided for on the tibial component, thereby to prevent the femoral component from rotating about an anterior-inferior axis.

The invention further provides that a peg formation may be located on the internal femoral attachment surface, preferably on the anterior member. Furthermore, the peg formation may be oriented to project along the longitudinal axis of the femur. The peg formation may thus define a further fixation point. The peg may be approximately 5 mm in length or longer and provision may be made for the peg to be extendable along its length.

The femoral internal surface may define a suitable surface texture, such as a roughened surface texture for facilitating bony tissue adhesion thereto, while the femoral articular bearing surface may have a highly polished surface for minimizing frictional contact. The roughened surface texture may be fine or rough surface irregularities, The roughened surface texture may further extend onto the surface of the peg formation. The femoral internal surface may further include a mesh, attached to and slightly proud of the femoral internal surface. This mesh may be made from plastics or metal, such as polyethylene or titanium, or a combination thereof.

There is further provided, according to the invention, that the femoral internal surface, inclusive of the peg formation, may be pre-treated or coated with hydroxyapatite, or any suitable growth material, prior to or during surgical placement of the femoral component.

The invention may further provide for the femoral internal surface to have at least one rib formation extending from the flattened interior surface to the peg formation. Preferably the formation is centrally located on the femoral interior surface and extends from the flattened interior surface to just beyond the peg formation. Preferably, the rib may have a height of 1 mm at its highest point and may have a roughened surface and/or mesh.

The femoral component may be made from any suitable material, including but not limited to metal, ceramic, peek, or a blend of these, or a polyethylene, such as ultra high molecular weight polyethylene. Preferably, the femoral component may be made from a metal alloy, such as a surface modified titanium alloy.

According to a further aspect of the present invention, there is provided a revision method of re-surfacing chondral deficient surface areas in the knee joint, wherein the method is minimally invasive, including the step of utilizing and inserting an orthopedic prosthesis including a femoral component, substantially as herein described.

### BRIEF DESCRIPTION OF THE DRAWINGS

The invention will now be described in greater detail, by way of non-limiting example, with reference to the following drawings, in which:

FIG. 1 shows a schematic perspective view of an improved knee prosthesis in situ, according to one form of the present invention;

FIG. 2 shows a schematic cross-sectional front view of the anterior member of femoral component of the knee prosthesis of FIG. 1;

FIG. 3 shows a schematic perspective view of the knee prosthesis of FIG. 1, viewed from an opposite (inverted) angle;

FIG. 4 shows a schematic bottom view of the femoral component of the knee prosthesis of FIG. 1;

FIG. 4(A) shows a schematic cross-sectional side view of the knee prosthesis of FIG. 4 through the line A-A′;

FIG. 4(B) shows a schematic cross-sectional side view of the knee prosthesis of FIG. 4 through the line B-B′;

FIG. 4(C) shows a schematic cross-sectional side view of the knee prosthesis of FIG. 4 through the line C-C′;

FIG. 5 shows a schematic perspective top view of the knee prosthesis of FIG. 1;

FIG. 6 shows a schematic side view of the knee prosthesis of FIG. 1;

FIG. 7 shows a schematic perspective view of the knee prosthesis of FIG. 1, viewed from an opposite angle; and

FIG. 8 shows a schematic perspective view of the knee prosthesis of FIG. 1, viewed from a different angle wherein the femoral upper bearing surface is illustrated; and

FIG. 9 shows a schematic top perspective view of the knee prosthesis of FIG. 5, including a mesh located on the femoral internal attachment surface.

### DETAILED DESCRIPTION OF THE INVENTION

In the drawings, like numerals refer to like parts, unless otherwise indicated.

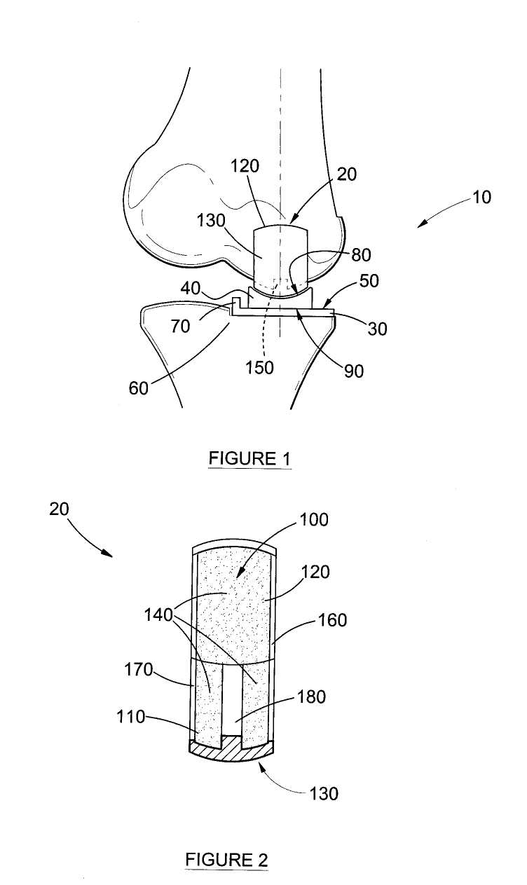

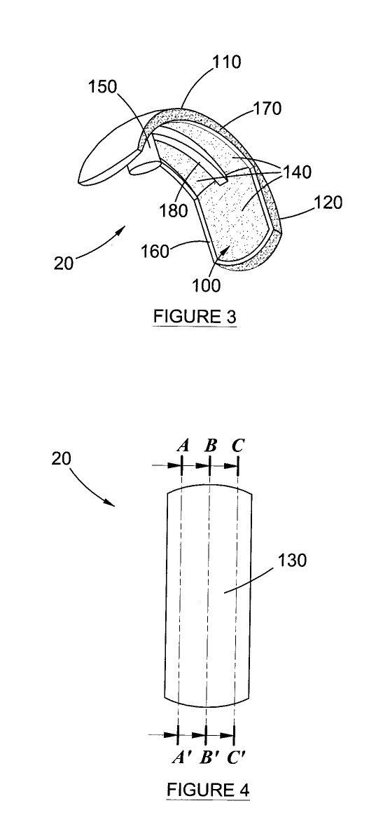

Referring firstly to FIG. 1, reference numeral 10 generally refers to an improved orthopedic prosthesis according to one form of the present invention. The orthopedic prosthesis 10 comprises a femoral component generally indicated by reference numeral 20, and a tibial component 30. A spacer bearing 40 is positioned between the femoral component 20 and the tibial component 30.

The tibial component 30 is of the type conventionally utilized in the art and consists of a tibial upper bearing surface 50 in a substantially planar D-shape when viewed in plan view (not shown). The tibial planar upper bearing surface 50 receives the spacer bearing 40 in a floating arrangement. Typically the tibial upper bearing surface 50 is highly polished in order to optimize free movement of the spacer bearing 40 thereon and to reduce friction therebetween. The tibial component 30 further has a lower attachment surface (not shown) for attachment to the tibial plateau 60. A tibial anchor (not shown) in the form of a median fin extending across the tibial lower attachment surface (not shown) relative to the tibial component 30 enhances anchorage of the tibial component 30 to the tibial condyle or tibial plateau 60. These are features that are known in the art. A further feature of the tibial component 30 is an orthogonal lip 70 located on the medial edge of the tibial component 30 for providing a further abutment surface for the spacer bearing 40.

Typically, the spacer bearing 40 is made of a compressed polyethylene manufactured by way of direct compression moulding, as is known in the art. The spacer bearing 40 has an upper bearing surface 80, spherically shaped concavely when viewed in plan view, to accommodate the femoral component 20, as will be described in further detail below. The spacer bearing 40 further has a planar lower bearing surface 90 for movable abutment with the tibial upper bearing surface 50.

The femoral component 20 has a c-shaped, uni-radial, elliptical body when viewed from the side, as can be seen more clearly in FIG. 6. The femoral component 20 has an anterior member 110 and a posterior member 120. The anterior member 110 and the posterior member 120 collectively serve to define the aforementioned elliptical body. The femoral component 20 further has a highly polished femoral articular bearing surface 130 and a femoral internal attachment surface 140. The femoral articular bearing surface 130 engages the upper bearing surface 80 of the spacer bearing 40.

The posterior member 120 defines a flattened posterior (internal) surface 100, which is an extension of the femoral internal attachment surface 140. The elliptical body, and in particular, the femoral internal attachment surface 140 (together with the flattened posterior surface 100) is anatomically shaped to facilitate placement of the femoral component 20 without a significant amount of bone removal.

With reference to FIG. 2, the femoral internal attachment surface 140 and the femoral articular bearing surface 130 have a substantially uniform cross-sectional curvature at any point along the length of the anterior member 110. The anterior member 110 has a thickness of 2 mm, along its entire length.

The elliptically shaped femoral component 20 thus provides a free range of movement of approximately 0° to 130° in situ.

The femoral internal attachment surface 140 has a peg formation 150 located on and extending upwardly from the femoral internal attachment surface 140 of the anterior member 110. The peg formation 150 is oriented to project in a direction along the longitudinal axis of the femur, as can be seen in FIG. 1. In the illustrated embodiment, the peg has a basic length of 5 mm. This height is extendable by virtue of a screw and screw threaded bore (not shown) located within the peg formation 150, of the conventional type.

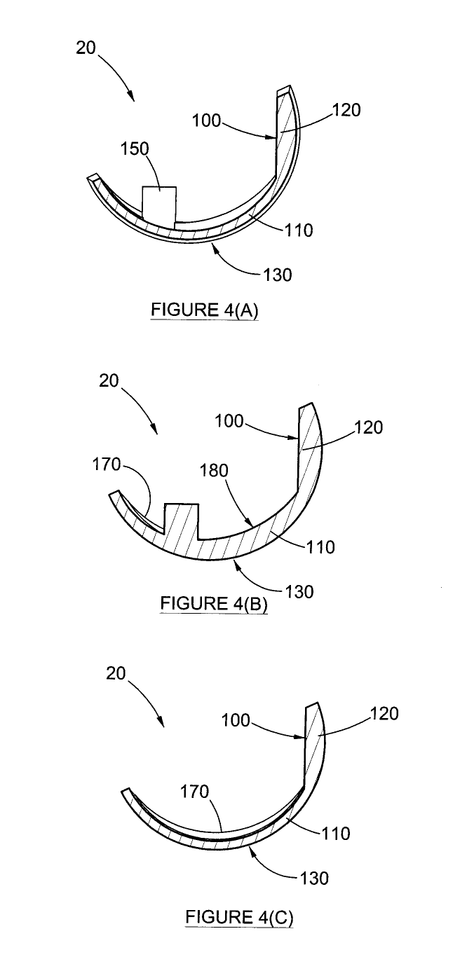

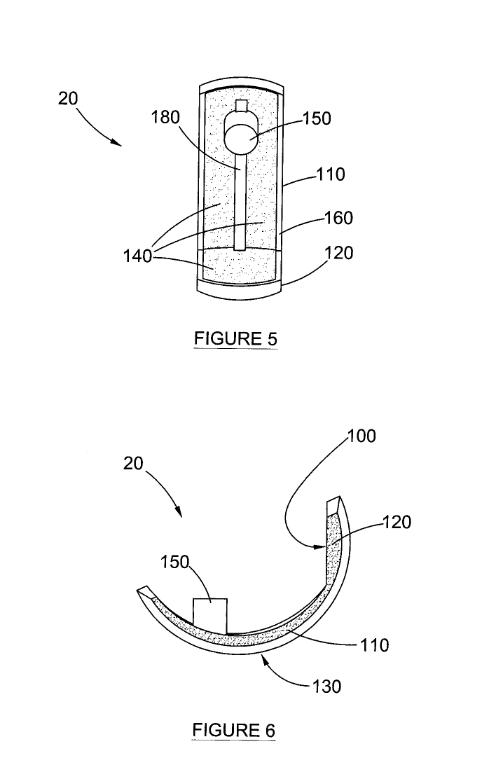

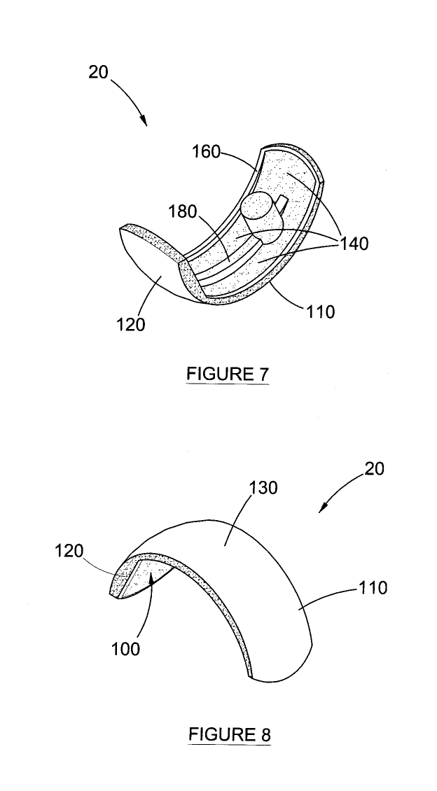

A further line of attachment 160 extends across the substantially outer circumferential edge of the femoral internal attachment surface 140. Furthermore, the femoral component 20 has a medial edge 170 as can be seen most clearly in FIGS. 4(B) and 4(C). This medial edge 170 movably abuts the orthogonal lip 70 on the tibial component 30.

The femoral internal attachment surface 140 and the peg formation 150 have fine surface irregularities for facilitating bony tissue adhesion thereto, in contrast to the femoral articular bearing surface 130 which has a highly polished surface. The internal attachment surface 140, and the peg formation 150 is coated with hydroxyapatite.

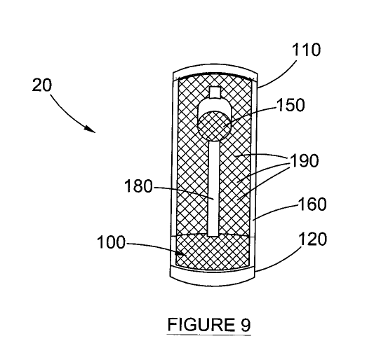

Furthermore, the femoral internal attachment surface 140 has a single, centrally located rib formation 180 extending from the flattened posterior surface 100 to the peg formation 150, and extends just beyond the peg formation 150. This rib formation 180 has a height of 1 mm at its highest point and also has fine surface irregularities. In addition, as can be seen from FIG. 9, the femoral internal attachment surface 110 has a metal mesh 190 located on and slightly proud of the femoral internal attachment surface 110.

The femoral component 20 is made from a surface modified titanium alloy.

In use, in a surgical setting, an orthopedic surgeon exposes the medial aspect of the knee for purposes of knee joint revision, as set out herein. While maintaining the integrity of the collateral and cruciate ligaments, and depending on the severity of damage to the surrounding tissue, the medial meniscus is surgically removed. A sufficient amount of bone is also removed from the tibial plateau 60 in order to accommodate the tibial component 30. Further bony incisions are made, as may be necessary, and as determined by the particular tibial component 30 used.

The tibial component 30 is then placed onto the revised tibial plateau 60 and secured thereon using any suitable securing and/or attachment means as is known in the art. Typically, this involves the use of bone cement and may alternatively be cementless, or a combination of these.

The medial femoral condyle is the next subject of revision. Here, bone is removed in order to accommodate the femoral component 20. A bore, aligned with the longitudinal axis of the femur is also introduced in the femoral condyle. The femoral component 20, suitably sized (in small, medium, large or extra large, as the case may be), pre-treated with hydroxyapatite, is then placed onto the area of revision on the femoral condyle, and secured thereto using bone cement (or may be cementless). The aforementioned bore receives the peg formation 150 of the femoral component 20. A suitably sized polyethylene plastic spacer 40 is selected and positioned between the femoral component 20 and the tibial component 30. The incision is surgically closed and allowed to heal. Radiographs may be taken from time to time in order to assess the success of the aforementioned procedure during the healing period.

A person skilled in the art will appreciate the following advantages posed by the present invention, namely, four points of attachment that are of importance and are inherent in the design of the femoral component 20. The most important of these are the increased curvature of the anterior member 110 of the femoral component 20 resulting in an internal surface area increase, which has the advantage of offering a greater cross-sectional line of attachment to bony tissue and hence an increased contact surface when compared to the prior art devices.

A second attachment point relates to the flattened posterior surface 100, which on its own functions to prevent rotation of the femoral component 20 while in situ.

A third attachment point, which may work in concert with the second attachment point, relates to the peg formation 150.

A fourth attachment point which enhances the strength of attachment relates to the circumferential line of attachment 160 located on the outer periphery of the internal femoral attachment surface 140 of the femoral component 20.

These attachment points in combination seek to retain the femoral component 20 in site and reduces the probability of aseptic loosening of the femoral component 30, which would result in a failed insert.

A further advantage posed by the femoral component 30 is the reduction in ramp height, without the concomitant removal of a significant amount of bone from the femoral condyle. Ramp height as used in this context means the distance between the femoral condyle and the tibial plateau. This results in less strain being applied to the knee ligaments, especially the cruciate ligaments. Furthermore, by mimicking the natural ramp height of a healthy knee, the right amount of tension will be maintained in the collective knee ligaments, thereby preventing undue pressure being exerted on each respective bearing surface (50, 90, 80 and 130), in the orthopedic prosthesis 10.

With the reduced amount of bony tissue removed from the femoral condyle, the possibility of a further prosthetic replacement, without the resort to a more invasive complete knee replacement, is still viable.

Although certain forms of the invention only have been described herein, it will be understood by any person skilled in the art that other modifications or variations of the invention are possible. For instance, the rib formation 180 located on the femoral internal attachment surface 140 need not be a single rib, and in fact may comprise more than one rib. Further, the peg formation 150 need not be a single peg formation, as more than one peg formation can be used herein. Furthermore, even though the preferred embodiment describes use of the present invention in the case of anteromedial arthritis, the inventor foresees the possibility that the invention may work equally well for lateral femoral condyle resurfacing, or a combination of both medial and lateral resurfacing techniques, as well.

Such modifications and/or variations are therefore to be considered as falling within the spirit and scope of the present invention as herein described.

1.-25. (canceled)

26. An orthopedic prosthesis suitable for resurfacing chondral deficient surface areas in knee joints, including a tibial component defining a tibial articular bearing surface, a femoral component defining a femoral articular bearing surface and a spacer bearing disposed therebetween, the femoral component further including:
an elliptical body having an anterior member and a posterior member, and an internal femoral attachment surface;
the femoral articular bearing surface and the internal femoral attachment surface having a substantially uniform cross-sectional curvature at any point along the length of the anterior member, thereby providing an increased radius of the internal femoral attachment surface and an increased cross sectional line of fixation to bony tissue; and
the internal femoral attachment surface further defining a line of attachment extending across the substantially outer circumferential edge thereof.

27. The orthopedic prosthesis as claimed in claim 26, wherein the elliptical body is a substantially c-shaped body when viewed from the side.

28. The orthopedic prosthesis as claimed in claim 26, wherein the elliptical body is a uniradial body.

29. The orthopedic prosthesis as claimed in claim 26, wherein the elliptical body provides a free range of movement of approximately 0° to 130° in situ.

30. The orthopedic prosthesis as claimed in claim 26, wherein the cross-sectional thickness of the anterior member is less than 3 mm thick, and is preferably 2 mm thick, at any point along the anterior member.

31. The orthopedic prosthesis as claimed in claim 26, wherein the posterior member defines a flattened interior surface, thereby defining a posterior fixation point.

32. The orthopedic prosthesis of claim 26, wherein a peg formation is located on the internal femoral attachment surface, preferably on the anterior member.

33. The orthopedic prosthesis as claimed in claim 32, wherein the peg formation is oriented to project along the longitudinal axis of the femur.

34. The orthopedic prosthesis as claimed in claim 32, wherein the peg formation defines a further fixation point.

35. The orthopedic prosthesis as claimed in claim 26, wherein the peg has a length of 5 mm and is extendable along its length.

36. The orthopedic prosthesis as claimed in claim 26, wherein the femoral internal surface defines a suitable surface texture, such as a roughened surface texture for facilitating bony tissue adhesion thereto, while the femoral articular bearing surface has a highly polished surface for minimizing frictional contact.

37. The orthopedic prosthesis as claimed in claim 36, wherein the roughened surface texture is fine or rough surface irregularities, which extends onto the surface of the peg formation.

38. The orthopedic prosthesis of claim 1, wherein the femoral internal surface further includes a mesh, attached to and slightly proud of the femoral internal surface, and wherein the mesh surface is consistent with the shape of the femoral internal surface.

39. The orthopedic prosthesis as claimed in claim 38, wherein the mesh is made from plastics or metal, such as polyethylene or titanium, or a combination thereof.

40. The orthopedic prosthesis as claimed in claim 32, wherein the femoral is pre-treated or coated with hydroxyapatite, or any suitable growth material, prior to or during surgical placement of the femoral component.

41. The orthopedic prosthesis of claim 26, wherein the femoral internal surface has at least one rib formation extending from the flattened interior surface to the peg formation.

42. The orthopedic prosthesis as claimed in claim 41, wherein the rib formation is centrally located on the femoral interior surface and extends from the flattened interior surface to beyond the peg formation, and has a height of 1 mm at its highest point and has a roughened surface and/or mesh.

43. The orthopedic prosthesis as claimed in claim 26, wherein the femoral component is made from any suitable material, including but not limited to metal, ceramic, peek, or a blend of these, or a polyethylene, such as ultra high molecular weight polyethylene.

44. The orthopedic prosthesis as claimed in claim 26, wherein the femoral component is made from a metal alloy, such as a surface modified titanium alloy.

45. A revision method of re-surfacing chondral deficient surface areas in the knee joint, wherein the method is minimally invasive, including the step of utilizing and inserting an orthopedic prosthesis including a femoral component substantially as herein described.
